(19) United States
(12) Patent Application Publication
(10) Pub. No.: US 2025/0313869 A1
(43) Pub. Date: Oct. 9, 2025

(54) MONOGLYCERIDES FROM FERMENTATION AS FOOD PRESERVATIVES AND HEALTH SUPPLEMENTS

(71) Applicant: BioVeritas, LLC, Bryan, TX (US)

(72) Inventor: Cesar B. Granda, College Station, TX (US)

(73) Assignee: BioVeritas, LLC, Bryan, TX (US)

(21) Appl. No.: 19/240,593

(22) Filed: Jun. 17, 2025

Related U.S. Application Data
(63) Continuation of application No. PCT/US2023/084967, filed on Dec. 19, 2023.
(60) Provisional application No. 63/433,827, filed on Dec. 20, 2022.

(51) Int. Cl. C12P 7/6454; C07C 67/08; C07C 67/54; C12P 7/6409

(52) U.S. Cl. CPC C12P 7/6454; C07C 67/08; C07C 67/54; C12P 7/6409

(57) ABSTRACT

A method of preparing monoglycerides from fermentation-derived carboxylic and acids includes esterifying the fermentation-derived carboxylic acids with glycerol to produce the monoglycerides, wherein the monoglycerides are short-chain monoglycerides, medium-chain monoglycerides, or a combination of any two or more thereof.

### CROSS-REFERENCE TO RELATED PATENT APPLICATIONS

This application is a U.S. Continuation Application of International Application No. PCT/US2023/084967, filed Dec. 19, 2023, which claims the benefit of priority to U.S. Provisional Patent Application No. 63/433,827, filed on Dec. 20, 2022, both of which are incorporated herein by reference in their entirety.

### TECHNOLOGY

The methods disclosed herein are directed to producing derivatives from fatty acids generated from a fermentation process. More particularly the present technology is directed to the production of monoglycerides from carboxylic acids, and specifically to the production of short-fatty acids and medium-chain fatty acids generated from a mixed-culture fermentation process.

### BACKGROUND

Long-chain monoglycerides have been long used as emulsifiers in the food industry, such as in baking. There is rising interest in the use of short-chain monoglycerides and medium-chain monoglycerides for use as an anti-microbial. Short-chain monoglycerides and medium-chain monoglycerides are not pH dependent, allowing them to work well under either acidic or alkaline environments; and they are non-corrosive, which makes them inherently safer for both humans and animals and towards material assets used in their handling. There is a need for the economical and efficient production of short- and medium-chain monoglycerides.

### SUMMARY

Mixed-culture fermentations or anaerobic digestion use naturally occurring mixed organism consortia (a.k.a. mixed cultures or microbiomes) to digest a variety of biomass feedstocks, which produce mixed carboxylic acids when methanogenesis is inhibited or arrested. These carboxylic acids are short-chain fatty acids (SCFAs), such as acetic, propionic, butyric, iso-butyric acids, valeric and iso-valeric acids, (C₂-C₅) and medium-chain fatty acids (MCFAs), such as caproic, heptanoic, caprylic, and nonanoic acids (C₆-C₉). More advanced controls of mixed-culture fermentation allow control of the chain length. Controlling certain conditions such as pH, temperature, certain additives and feedstock composition, allow for production of mainly SCFAs or for chain elongation to yield mostly MCFAs. Such acids can be employed as a reactant to produce short- and medium-chain monoglycerides (SCMs and MCMs, respectively) by esterification of the fatty acids with glycerol. The esterification may be carried out without any catalysts or using catalysts such as homogeneous or heterogeneous solid acid or base catalysts and enzymes. Thus, SCFAs and MCFAs may be reacted to produce short-chain monoglycerides (SCMs) and medium-chain monoglycerides (MCMs) with a high degree of selectivity and control. Further, as the process can rely on the fermentation of natural, biodegradable feedstock for the production of the SCFAs and MCFAs, the SCMs and MCMs may be produced without the use of petroleum-derived feedstocks, which may give the resulting products an advantage in the marketplace. The produced SCMs and MCMs will have a high (90%+) amount of bio-based carbon.

In one aspect, a method of generating monoglycerides is provided. In some embodiments, the method includes reacting carboxylic acids to produce the monoglycerides, wherein the carboxylic acids are produced by fermenting a biodegradable feedstock to produce a fermentation product stream comprising the carboxylic acids, and wherein the monoglycerides are short-chain monoglycerides, medium-chain monoglycerides, or a combination thereof. In some embodiments, reacting the carboxylic acids includes direct esterification of the carboxylic acids with glycerol without a catalyst. In other embodiments, reacting the carboxylic acids includes direct esterification of the carboxylic acids with glycerol in the presence of an acid catalyst. In other embodiments, reacting the carboxylic acids includes direct esterification of the carboxylic acids with glycerol in the presence of a base catalyst. In other embodiments, reacting the carboxylic acids includes direct esterification of the carboxylic acids with glycerol in the presence of an enzyme as catalyst.

In some embodiments, the fermentation used to produce the fatty acids is a mixed-culture fermentation, or an anaerobic digestion. In some embodiments, the fermentation is of a biodegradable feedstock. In some embodiments, after reacting the carboxylic acids the resulting monoglycerides are purified. In some embodiments, the purifying is performed by vacuum distillation, vacuum steam distillation, molecular distillation, or combinations thereof. In some embodiments, the method includes a fermentation step to generate a fermentation product stream including carboxylic acids. In some embodiments, the carboxylic acids undergo a recovery step in which the carboxylic acids are recovered from a fermentation product stream. In some embodiments, a by-product stream from the reaction of the carboxylic acids and/or the purification step may be recycled to the fermentation step or to the carboxylic acid recovery step.

In some embodiments, short-chain fatty acids with a carbon length from C₂ to C₅ or combinations thereof are reacted to produce short-chain monoglycerides. In some embodiments, medium-chain fatty acids with a carbon length from C₆ to C₉, or combinations thereof, are reacted to produce medium-chain monoglycerides. The carboxylic acids that are reacted to produce medium-chain monoglycerides are medium-chain fatty acids, or combinations thereof. In some embodiments, the final product may be a mixture of monoglycerides and glycerol and small amount of di-glycerides and triglycerides. In other embodiments, the glycerol may be removed to increase the concentration of the monoglycerides.

In another aspect, a preservative including monoglycerides is provided, wherein the monoglycerides are short-chain monoglycerides, medium-chain monoglycerides, or a combination thereof, and wherein the monoglycerides are prepared by reacting carboxylic acids obtained from a fermentation process with glycerol.

In another aspect, a method of using a preservative is provided, where the method includes using the preservative as an antimicrobial preservative, an antifungal preservative, a texture modifier, a surfactant emulsifier, a nutritional additive, a dietary supplement, or combinations thereof, wherein the preservative comprises short-chain monoglycerides, medium-chain monoglycerides, or a combination thereof, and wherein the monoglycerides are prepared by reacting carboxylic acids obtained from a fermentation process with glycerol. The short- and medium chain monoglycerides may be used as preservatives in human and animal food, surfactants/emulsifiers, texture and food property modifiers, and can at the same time serve as nutritional additives, dietary supplements, or both.

In another aspect, provided herein is a method of preparing monoglycerides, the method including: fermenting a biodegradable feedstock to produce a first fermentation broth including short- and medium-chain fatty acids; recovering at least a portion of the short- and medium-chain fatty acids in the fermentation broth; esterifying the fermentation-derived carboxylic acids with glycerol to produce monoglycerides; and purifying the monoglycerides to produce a product stream including purified monoglycerides and a by-product stream comprising unreacted glycerol, unreacted fermentation derived carboxylic acids, or a combination of any two or more thereof; recycling the by-product stream to one or more of the fermenting step and the short- and medium-chain fatty acid (carboxylic acid) recovering step, wherein the monoglycerides are short-chain monoglycerides, medium-chain monoglycerides, or a combination of any two or more thereof.

In some embodiments, the method includes at least one of conditioning the fermentation broth before recovering and fractionating the fermentation-derived carboxylic acids before esterifying. In some embodiments, the esterifying is carried out in the presence of an acid catalyst, a base catalyst, or an enzyme catalyst. In some embodiments, the produced monoglycerides have greater than 90% bio-based carbon content as measured by ASTM D6866.

### BRIEF DESCRIPTION OF THE DRAWING

FIG. 1 is a schematic showing an integration of a fermentation process with direct esterification, according to an embodiment.

### DETAILED DESCRIPTION

Various aspects and embodiments are described herein. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As utilized herein with respect to numerical ranges, the terms “approximately,” “about,” “substantially,” and similar terms will be understood by persons of ordinary skill in the art and will vary to some extent depending upon the context in which it is used. If there are uses of the terms that are not clear to persons of ordinary skill in the art, given the context in which it is used, the terms will be plus or minus 10% of the disclosed values. When “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Described herein are systems and methods for converting carboxylic acids from fermentation to short-chain and medium-chain monoglycerides. Also described herein are preservatives produced from short-chain and medium chain monoglycerides and methods of use of those preservatives.

It should be understood that, although example implementations of embodiments of the disclosure are described herein, the systems, methods, and uses of this disclosure may be implemented using any number of techniques, whether currently known or not. The present disclosure should in no way be limited to the example implementations, and techniques illustrated below.

It is also noted that the “process streams” described herein need not be clean cut or pure. When referring to particular reactant and product streams herein, it should be understood that, although the primary product(s) may be described, other products may exist in the streams. Thus, there may be quantities of the other compounds in such streams and/or other impurities.

The methods disclosed herein are related to the production and uses of monoglycerides having the following structure:

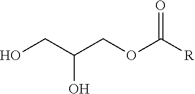

wherein R is an alkyl group. The structure show above are 1-monoglycerides, which would be more common due to less steric hindrance, however, 2-monoglycerides can also form with the following structure:

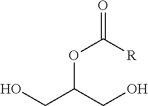

wherein R is an alkyl group,

Short-chain monoglycerides (SCMs) are monoglycerides where R is a short-chain alkyl of 1, 2, 3, or 4 carbon length (C₁, C₂, C₃, and C₄, respectively) or combinations thereof. Medium-chain monoglycerides (MCMs) are monoglycerides where R is a medium-chain alkyl of 5, 6, 7, 8, 9, 10, or 11 carbon length (i.e., C₅, C₆, C₇, C₈, C₉, C₁₀ or C₁₁ respectively) or combinations thereof.

For mixed-culture fermentation-derived acids, the alkyl groups typically range from C₂ through C₉ fatty acids. These acids may be converted by monoglycerides by the method described herein, reacting glycerol with a fatty acid having the desired tail length. The final product of the methods presented herein may be a mixture having primarily monoglycerides and other components. For example, the final product stream may include diglycerides and/or triglycerides. The final product may also include unreacted glycerol. However, the product may still be referred to as monoglycerides (either SCMs or MCMs) even though diglycerides, triglycerides and glycerol may also be present in some quantities.

### Production of Short- and Medium-Chain Fatty Acids

Mixed-culture fermentation (also known anaerobic digestion) from renewable biomass resources is one of the most economically competitive methods to convert biomass materials to renewable chemicals and fuels. These microorganisms generally produce a mixture of carboxylic acids, which are short- and medium-chain fatty acids (SCFAs and MCFAs) ranging from acetic acid (C₂) to nonanoic acid (C₉). Specifically, carboxylic acids of all chain lengths from C₂ and C₉ may be produced by the fermentation. For example, propionic, butyric, iso-butyric, valeric, caproic, heptanoic, octanoic, and nonanoic acids may be produced. These same microorganisms used in the fermentation process also produce the same carboxylic acids in the human and animal gastrointestinal tract (den Besten et al. 2013). Through controlled fermentation (temperature, pH, volatile solids loading rate, liquid residence time), selective recovery, and with or without the addition of certain additives or reducing compounds like ethanol or hydrogen (Kenealy et al. 1995, Agler et al. 2012), the product profile of the carboxylic acid products can be adjusted to produce short or medium carboxylic acids. Typical temperatures that are used in mixed-culture fermentation may be from 35 to 60° C. The pH of the fermentation process is important, as it will also affect the final profile, however, too low of a pH will inhibit microorganisms, especially due to the fact that most organic acids are toxic at lower pH's. Thus, pH may be from 5 to 9, but preferably is maintained from 6 to 7.

In addition, another parameter to control is the volatile solids loading rate (VSLR, the rate at which volatile solids, which is a proxy organic matter, is fed into the fermentation) and the hydraulic or liquid residence time (LRT). Longer residence times will select for slower growing microorganisms and vice versa; shorter residence time will select for faster growing ones. Typical VSLRs are from 6 to 24 g volatile solids/(L−day), and preferably 8 to 23 g volatile solids/(L−day). The residence time, on the other hand can be from less than 1 day all the way to 32 days, but preferably 2 to 10 days.

As the carboxylic acids are produced by a fermentation of biomass, the carboxylic acids and resulting monoglycerides are formed of bio-based materials. The carboxylic acids and monoglycerides produced by the methods disclosed herein are substantially free of any petroleum-based materials. The amount of bio-based material in a carboxylic acid sample can be ascertained by measuring the amount of ¹⁴C in the sample, with higher levels of ¹⁴C indicating that the carbon in the sample is from recent biological sources. Lower levels of ¹⁴C indicate that the carbon in the sample is older, and is therefore likely to have been sourced from petroleum, natural gas, coal, or other such deposits. For example, the bio-based materials may have a ¹⁴C content of greater than 0.01 ppt (parts per trillion). This includes from about 0.1 to about 1.1 ppt, or about 1 ppt. Petroleum based materials will have ¹⁴C content of less than about 0.01 ppt. The amount of ¹⁴C may be determined according to ASTM Standard D6866. The carboxylic acids produced by the methods disclosed herein may have greater than 90% bio-based carbon, as measured by ASTM Standard D6866.

The fermentation broth may optionally be treated to remove impurities and solids. Solids suspended in the fermentation broth may be filtered out, or the broth may be subjected to centrifugation to remove suspended solids. Conditioning of the broth may be done through physical or chemical processes, or through a combination. For example, conditioning of the broth may be accomplished by techniques known in the art, such as through the use of settling tanks or membrane filtration. The level of filtration will depend on the particle size of the suspended solids and the desired clarity of the resulting broth, but it should be understood that microfiltration, ultrafiltration, and nanofiltration filters may be employed to remove small, suspended solids from the broth. In addition or alternatively, the fermentation broth may be concentrated by processes such as reverse osmosis or evaporation to remove water and other volatile impurities, such ammonia. Addition of clarifying agents may be useful for causing flocculation of solids suspended in the broth.

The carboxylic acids may be recovered from a fermentation broth (whether raw or conditioned) using several methods, such as acidification and extraction, followed by distillation (Ross and Granda U.S. Pat. No. 10,662,447) in a carboxylic acid recovery system (CARS). These carboxylic acids are high-value products in the chemical market for applications such as in food and feed additives, detergents, cosmetics, food additives, paints, lubricants, plasticizers and among others. However, because markets are limited, it is of great interest to find more opportunity for growth.

Monoglycerides containing short- and medium-chain fatty acids have potentially significant application as preservatives that can act also as nutritional additives and dietary supplements for gut health in humans and animals (Righi et al., 2020; Jackman et al. 2020; De Keyser, et al 2019) and this is because, as mentioned, these same carboxylic acids (C₂ through C₈, but especially C₂ through C₄) are also produced by beneficial microorganisms in the gut (den Besten et al. 2013; Ríos-Covián et al.); therefore, getting these carboxylic acids to the gut, especially to the lower parts of the gastrointestinal tract, brings many health benefits (Jackman et al. 2020). Their delivery as glycerides ensures that the carboxylic acids are not degraded or absorbed along the way so that they may indeed reach the lower part of the gastrointestinal tract (Ploegmakers et al. 2019).

After the carboxylic acids have been recovered, they may be optionally fractionated to purify them and to produce substantially pure acid fractions including short-chain and/or medium-chain carboxylic acids. In some embodiments, an acid fraction may contain a single class of acids (i.e., short-chain or medium chain fatty acids). In some embodiments, the acid fractions may substantially comprise an individual acid (e.g., propionic acid or butyric acid). Fractionation may be accomplished through, as a non-limiting example, distillation of the recovered carboxylic acids. Fractions containing short-chain and/or medium-chain carboxylic acids can be sent to the esterification step of the process.

### Production of Short- and Medium-Chain Monoglycerides

A method to make glycerides is direct esterification of glycerol and carboxylic acids. For example, mono-, di-, and triglycerides can be produced through catalytic reaction of glycerol and corresponding fatty acids using a catalyst as shown below in Scheme 1.

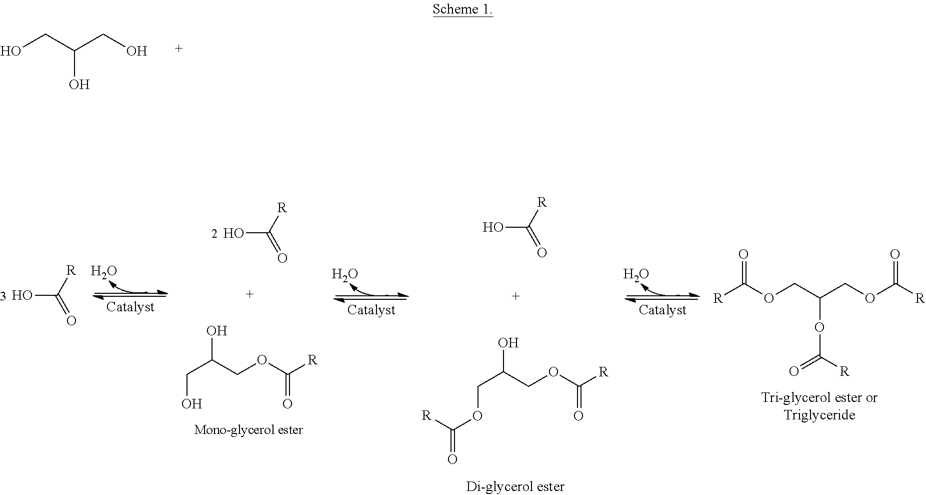

The catalysts for esterification of the fatty acids with the glycerol can be homogeneous type or heterogeneous type. Homogeneous acids such as sulfuric or sulphonic acids are typically used for esterification of carboxylic acids and glycerol (Mostafa et al. 2013), but bases can also be employed (Younes et al. 2017). Solid acids of either Bronsted type or Lewis type acids are reviewed for glycerol acetylation (Kong et al. 2016). Specifically, esterification of glycerol and fatty acids have been studied under reduced pressure with assistance of various metal chlorides and oxides as catalysts. For example, zirconia-supported hetero-polyacid catalyst (HSiW/ZrO₂) was reported to make glyceryl diacetate or triacetate. Highly acidic sulfonated zirconia catalyst (SO₄²⁻/ZrO₂) was reported as more efficient for esterification of glycerol in excess of acetic acid. Enzymatic esterification of fatty or carboxylic acids has also been performed, such as using certain lipase enzymes, such as Novozyme lipase enzyme 435, but this is not effective with SCFAs, but it works well with MCFAs or larger (C6 and above).

### Purification

During these conversions, heterogeneous (i.e., solid) catalysts, such as enzymes, may be removed by filtration, which allows recycle of the catalyst. Also, consideration about using the solid catalyst in a packed bed must also be given. The final monoglyceride products will also require further purification to remove unreacted raw materials, such as unreacted fatty acids, water and fatty acid salts (soaps formed when an alkali is used as a homogeneous catalyst) and, if required, glycerol. Many times, these removed residues can be recycled back. For instance, unreacted glycerol may be returned to the esterification process. Free fatty acids and water may not be recycled back to the esterification step, as the water must be removed during esterification and SCFAs and MCFAs form azeotropes with water. For MCFAs, lowering the temperature of the azeotrope will allow the MCFAs separation from the water, allowing recycle of some of the acid. However, SCFAs are more soluble or fully soluble in water and therefore cannot be recovered by simply lowering the temperature. The water containing the acids at a high concentration (>20 g/L) may be recycled to the CARS used to recover the acids from fermentation broth when integrated with fermentation as described above. If the acid concentration is low (<20 g/L), then the water should be recycled to the fermentation process instead of CARS.

By-product streams from the purification step can also be recycled back to the fermentation step as a fermentation feedstock. For example, any waste glycerides can be recycled to the fermentation, where microorganisms are able to digest them and convert them back into carboxylic acids. Additionally, excess unreacted glycerol from the esterification reaction collected in the purification step can be fed to the fermentation step, where it can be converted to carboxylic acids.

Further refinement of the product may occur by bleaching the product with washes with, but not limited to, dilute phosphoric acid, with sodium bicarbonate solution to neutralize and remove any left-over unreacted acids and with water. Finally, the product may be passed through, for example, but not limited to, a bleaching clay or earth. If necessary, further impurities may be removed by passing the product through, for example, but not limited to, activated carbon. The by-product streams may be considered for recycle to the fermentation or to the carboxylic acid recovery system (CARS).

### Integrated Process

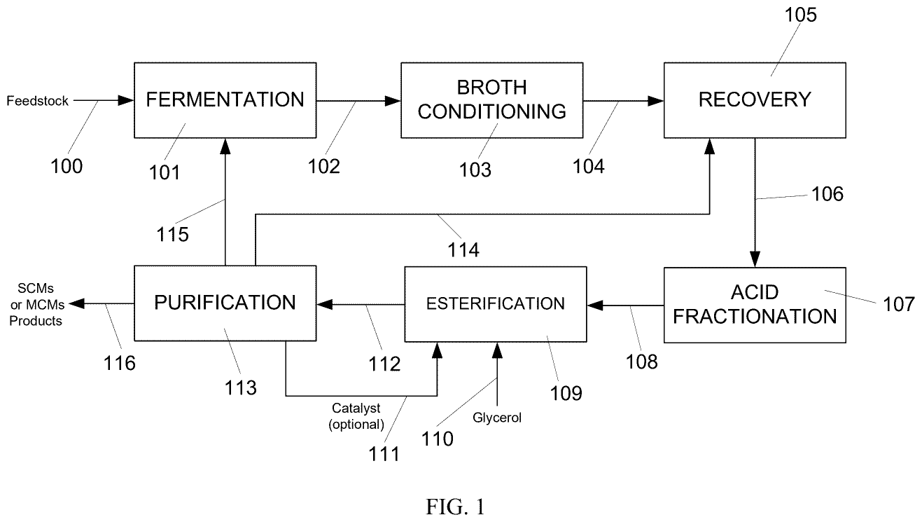

With reference to FIG. 1, an integrated process for the production of SCMs and/or MCMs may include a fermentation step (101), an optional broth conditioning step (103), an acid recovery step (105), an optional acid fractionation step (107), an esterification step (109), and a purification step (113).

As illustrated in FIG. 1, a biodegradable feedstock (100) may be fed to the fermentation process (101), which produces carboxylic acids (short- and medium-chain fatty acids, 102). In some embodiments, the biodegradable feedstock (100) may be, but is not limited to, a starch-based feedstock such as corn, wheat, oats, or cellulosic such as sugarcane bagasse, corn stover, straw, citrus peels, or mixtures of any two or more thereof. It should be understood that other biodegradable feedstock material may be used, either separately or in addition to those already provided. The biodegradable feedstock (100) is fermented in mixed culture fermentation or anaerobic digestion, in which microorganisms convert the biodegradable feedstock into mixed carboxylic acids (e.g., short- and medium-chain fatty acids), resulting in a fermentation broth (102).

The fermentation broth (102) containing the acids or salts of the acids may optionally undergo a conditioning step (103). In the conditioning step (103), the fermentation broth (102) may be settled, filtered, or subjected to centrifugation to remove solids from the broth. In addition or alternatively, in the conditioning step (103), the fermentation broth (102) may be subjected to membrane filtration (such as nanofiltration, ultrafiltration, microfiltration) to remove small, suspended solids. In addition or alternatively, in the conditioning step (103), the fermentation broth (102) may be concentrated by processes such as reverse osmosis or evaporation to remove water and other volatile impurities, such ammonia. The conditioned broth (104) may then be sent to a carboxylic acid recovery system (CARS) (105) as previously described, where the acids are recovered from the water.

The recovered mixed carboxylic acids (comprising short- and medium-chain fatty acids) (106) may be optionally sent to acid fractionation (107) to purify them and produce substantially pure acid fractions including short-chain and/or medium chain carboxylic acids. In some embodiments, an acid fraction may contain a single class of acids (i.e., short-chain or medium chain fatty acids). In some embodiments, the acid fractions may substantially comprise an individual acid (e.g., propionic acid or butyric acid).

A stream comprising the carboxylic acids (108) may be sent to esterification (109). In the esterification process (109), carboxylic acids are reacted with glycerol (110). The stream sent to esterification may have undergone the recovery process and/or the acid fractionation process, but need not have undergone either of these processes. The esterification (109) may be performed in the presence of catalysts (111) or without. In some embodiments, the catalyst (111) is an acid catalyst. In some embodiments, the catalyst (111) is a base catalyst. In some embodiments, the catalyst (111) is an enzymatic catalyst. Catalysts that can be optionally used, as non-limiting examples, may include lipase enzymes, acids, solid acids catalysts, alkalis such as, but not limited to potassium hydroxide or sodium hydroxide, and other salts.

To maximize the production of monoglycerides, and minimize further reactions to di- and triglycerides, a stoichiometric excess of glycerol is provided to the reactor, thereby minimizing di- and tri-esterification of the glycerol. The carboxylic acids react with the glycerol to form short-chain (SCMs) and/or medium-chain monoglycerides (MCMs), which can be extracted as a product stream (112).

The resulting product stream from the esterification (112) is further sent to purification (113) where it undergoes clean up as described above. In embodiments in which a catalyst (111) is used, the catalyst may also be recovered in the purification step (113) and, if possible, may be recycled to the esterification step (109) for further use. Other impurities, such as unreacted carboxylic acids or their salts (114) may be recycled back to the carboxylic acid recovery system (CARS) (105). In some embodiments, excess glycerol may be separated in the purification stage (113) and fed back to the esterification step (109), or may be sent as a recycle stream (115) to the fermentation step (101), as mixed-culture fermentation is able to digest glycerol and convert it to carboxylic acids. Other impurities separated from the esterification product stream (112), such as water, may also be included in the recycle stream (115), which may be recycled back to the fermentation (101). Finally, the final product stream (116) including SCMs or MCMs is obtained from the purification step. In some embodiments, the final product stream (116) includes small amounts of diglycerides, triglycerides, glycerol, or combinations of two or more thereof.

The methods disclosed herein may include the integration of a fermentation process, in which a biodegradable feedstock is fermented to produce carboxylic acids from acetic acid (C₂) to nonanoic acid (C₉) (i.e., short- and medium-chain fatty acids) with recovery of such carboxylic acids using a carboxylic acid recovery system (CARS), which efficiently recovers and purifies the acids from the effluent from the fermentation, and with further conversion of such acids to short- or medium-chain monoglycerides. However, the method disclosed herein may be performed independent of the fermentation process. For example, the methods disclosed herein may be performed on a fermentation broth purchased or otherwise acquired. Such a fermentation broth may require conditioning as previously described, or may already have been conditioned. Further, the methods described herein may be performed using a mixture of short- and/or medium-chain carboxylic acids as the beginning feedstock, starting with the fractionation step, if desired or required. The carboxylic acids may be directly fed to the esterification step per the methods disclosed herein.

Embodiments of the fermentation, also known as anaerobic digestion, typically use a mixed-culture of microorganisms, which ferment biodegradable feedstocks, which may be starch-rich, such as, but not limited to, corn-, oat-, or wheat-based feedstocks, or cellulosic feedstocks such as, but not limited to, sugarcane bagasse, corn stover, straw, and citrus peels. Such feedstocks may contain other components such as protein, ash, and fats.

In some embodiments, the pure individual acids or the mixed carboxylic acids prior to fractionation are reacted with glycerol in stoichiometric excess to effect esterification. Further, the product stream from this esterification may be purified using steam vacuum distillation or molecular distillation to remove unreacted acids, water, which can be recycled to CARS or to fermentation and unreacted glycerol, which may be recycled to the esterification step. The products may be short- or medium-chain monoglycerides, or combinations thereof, including some smaller quantities of di- and triglycerides and unreacted glycerol.

### Preservatives

The methods disclosed herein may be used to produce monoglycerides which may be used as preservatives. The monoglycerides may include short-chain and or medium-chain monoglycerides. The monoglycerides may include a fatty acid-derived chain that is from two to five carbons (C₂-C₅), from six to twelve carbons (C₆-C₁₂), from six to nine carbons (C₆-C₉), from six to eight carbons (C₆-C₈), from two to twelve carbons (C₂-C₁₂), from two to nine carbons (C₂-C₉), or from two to eight carbons (C₂-C₈). In some embodiments, the preservative includes monoglycerides of a single carbon chain length. For example, in some embodiments, the preservative includes monoglycerides having a carbon chain residue from the fatty acid that is 2, 3, 4, 5, 6, 7, 8, 9, 10, 11, or 12 carbon atoms in length. In some embodiments, the preservative includes a mixture of short-chain monoglycerides. In some embodiments, the preservative includes a mixture of medium-chain monoglycerides. In some embodiments, the preservative includes a short-chain monoglyceride. In some embodiments, the preservative includes a medium-chain monoglyceride. In some embodiments, the preservative includes both short-chain and medium-chain monoglycerides. All possible combinations of short- and medium-chain monoglycerides having from two to twelve carbons (C₂-C₁₂) are contemplated for use as a preservative, the monoglycerides being formed according to the methods previously disclosed herein.

The preservative may include substantially pure monoglycerides, or may alternatively include other compounds. For example, the preservative may include unreacted glycerol and diglycerides and triglycerides. In some embodiments, the preservatives are about 30 wt. % to about 90 wt. % monoglycerides. In some embodiments, the preservatives contain greater than about 40 wt. % monoglycerides. In some embodiments, the preservative contains from about 40 wt. % to about 80 wt. % monoglycerides. In some embodiments, the preservative contains from about 40 wt. % to about 70 wt. % monoglycerides. In some embodiments, the preservative contains from about 45 wt. % to about 60 wt. % monoglycerides.

In some embodiments, the preservative includes diglycerides and/or triglycerides. In some embodiments, the preservative contains from about 0.1 wt. % to about 10 wt. % diglycerides. In some embodiments, the preservative contains from about 0.5 wt. % to about 8 wt. % diglycerides. In some embodiments, the preservative contains from about 0.1 wt. % to about 10 wt. % triglycerides. In some embodiments, the preservative contains from about 0.1 wt. % to about 5 wt. % triglycerides. In some embodiments, the preservative contains from about 0.5 wt. % to about 3.5 wt. % triglycerides.

In some embodiments, the preservative also includes glycerol. In some embodiments, the preservative contains from about 10 wt. % to about 90 wt. % monoglycerides. In some embodiments, the preservative contains from about 20 wt. % to about 75 wt. % monoglycerides In some embodiments, the preservative contains from about 40 wt. % to about 65 wt. % monoglycerides In some embodiments, the preservative contains about 30 wt. % to about 90 wt. % monoglycerides, 0.1 wt. % to about 10 wt. % diglycerides, and 0.1 wt. % to about 5 wt. % triglycerides, with the balance of the preservative is substantially glycerol.

The preservatives described herein may act as an antimicrobial or as an antifungal. The preservatives described herein may also act as a texture modifier for foods. In some embodiments, the preservatives as described herein may be used as a surfactant emulsifier. The preservatives described herein may be a nutritional additive. The preservatives described herein may be a dietary supplement. In some embodiments, the preservatives described herein are dietary supplements for human consumption. In some embodiments, the preservatives described herein are dietary supplements for animal consumption, such as cows, fish, shrimp, pigs, chickens, turkeys, or any other type of livestock, or for other animals such as horses or deer. In some embodiments, the disclosed preservatives are added to an animal feed at a rate of about 0.1 wt. % to about 1.5 wt. %.

### REFERENCES

U.S. PATENT DOCUMENTS

2012/0029075

10,662,447

### OTHER PUBLICATIONS

Agler, M. T. et al., “Chain elongation with reactor microbiomes: Upgrading dilute ethanol to medium-chain carboxylates,” Energy Environ. Sci., 5 (8) (2012), 8189-8192
Beuchat, L. R. “Comparison of anti-vibrio activities of potassium sorbate, sodium benzoate, and glycerol and sucrose esters of fatty acids,” Applied and Environmental Microbiology, 39:6 (1980), 1178-1182
den Besten, G. et al “The role of short-chain fatty acids in the interplay between diet, gut microbiota, and host energy metabolism,” Journal of Lipid Research, 54 (2013), 2325-2340
De Keyser, K et al. “Medium-chain glycerides affect gut morphology, immune and goblet cells in post-weaning piglets: In vitro fatty acid screening with Escherichia coli and in vivo consolidation with LPS challenge,” J Anim Physiol Anim Nutr. 103 (2019), 221-230
Gomez-Osorio L. M et al “Short- and medium-chain fatty acids and their derivatives as a natural strategy in the control of necrotic enteritis and microbial homeostasis in broiler chickens,” Frontiers in Veterinary Science 8:773372 (2021), 1-11.
Bergsson, G et al. “In Vitro inactivation of Chlamydia trachomatics by fatty acids and monoglycerides,” Antimicrobial Agents and Chemotherapy 42:9 (1998), 2290-2294
Hyldgaard, M. et al. “Antimicrobial Mechanism of Monocaprylate,” Applied and Environmental Microbiology 78:8 (2012), 2957-2965
Jackman, J. A. et al. “Medium-chain fatty acids and monoglycerides as feed additives for pig production: towards gut health improvement and feed pathogen mitigation,” Journal of Animal Science and Biotechnology 11:44 (2020), 1-15.
Kaur, K. et al. “Facile Synthesis of Tributyrin Catalyzed by a Versatile Sulfated Iron Oxide: Reaction Pathway and Kinetic Evaluation,” Ind. Chem. Res. 55 (2016), 2534-2542
Kenealy W. R. et al. “Production of caproic acid by cocultures of ruminal cellulolytic bacteria and Clostridium kluyveri grown on cellulose and ethanol,” Appl Microbiol Biotechnol 44 (1995), 507-513
Kong P. S. et al. “Catalytic role of solid acid catalysts in glycerol acetylation for the production of bio-additives: a review,” RSC Adv., 6 (2016), 68885-68905
Kovanda, K. et al. “In Vitro antimicrobial activities of organic acids and their derivatives on several species of gram-negative and gram-positive bacteria,” Molecules 24 (2019), 3770-3784
Mostafa, N. A. et al. “Production of Mono-, Di- and Triglycerides from Waste Fatty Acids through Esterification with Glycerols,” Advances in Bioscience and Biotechnology, 4 (2013), 900-907
Namkung, H. et al. “Antimicrobial activity of butyrate glycerides toward Salmonella Typhimurium and Clostridium perfringens,” Poultry Science 90 (2011), 2217-2222
Ploegmakers, M. and Framelco R&D Team, “Glycerides of butyric acid: A must for poultry,” All About Feed, Feed Additives (2019) August 18th, Ed. Ploegmakers, M. (https://www.allaboutfeed.net/animal-feed/feed-additives/glycerides-of-butyric-acid-a-must-for-poultry/)
Ríos-Covián, D. et al. “Intestinal Short Chain Fatty Acids and their Link with Diet and Human Health,” Front Microbiol. (2016) 7, 185 (https://pubmed.ncbi.nlm.nih.gov/26925050/)
Righi, F et al. “Adding monoglycerides containing short and medium-chain fatty acids to milk replacer: effect on health and performance of preweaned calves,” Italian Journal of Animal Science 19:1 (2020), 1417-1427
Sun C. Q. et al. “Antibacterial actions of fatty acids and monoglycerides against Helicobacter pylori,” FEMS Immunology and Medical Microbiology 36 (2003) 9-17
Thormar H. et al “Stable concentrated emulsions of the 1-monoglyceride of capric acid (monocaprin) with microbicidal activities against the food-borne bacteria Campylobacter jejuni, Salmonella spp., and Escherichia Coli,” Applied and Environmental Microbiology, 7:1 (2006), 522-526
Younes, M. et al. “Scientific opinion on the re-evaluation of mono- and di-glycerides of fatty acids (E 471) as food additives,” EFSA Journal 15 (2017) 5045, 43 pp (https://doi.org/10.2903/j.efsa.2017.5045)

### EQUIVALENTS

Para. 1. A method of preparing monoglycerides from fermentation-derived carboxylic acids, the method comprising: esterifying the fermentation-derived carboxylic acids with glycerol to produce the monoglycerides, wherein the monoglycerides are short-chain monoglycerides, medium-chain monoglycerides, or a combination of any two or more thereof.

Para. 2. The method of Para. 1, wherein the glycerol is present in the esterifying in stoichiometric excess over the fermentation-derived carboxylic acids.

Para. 3. The method of Para. 1 or 2, wherein the esterifying is carried out without a catalyst or in the presence of a catalyst.

Para. 4. The method of Para. 3, wherein the esterifying is carried out in the presence of the catalyst comprising an acid catalyst.

Para. 5. The method of Para. 3, wherein the esterifying is carried out in the presence of the catalyst comprising a base catalyst.

Para. 6. The method of Para. 3, wherein the esterifying is carried out in the presence of the catalyst comprising an enzyme catalyst.

Para. 7. The method of any one of Para. 1-6 further comprising purifying the monoglycerides.

Para. 8. The method of Para. 7, wherein the purifying is performed by vacuum distillation, vacuum steam distillation, molecular distillation, or a combination of any two or more thereof.

Para. 9. The method of any one of Para. 1-8, wherein the fermentation-derived carboxylic acids are produced in a mixed-culture fermentation or an anaerobic digestion to produce a fermentation broth.

Para. 10. The method of Para. 9 further comprising recovering the fermentation-derived carboxylic acids from the fermentation broth in a carboxylic acid recovery process.

Para. 11. The method of Para. 10, wherein one or more by-product streams of the purification are recycled to the fermentation step or to the carboxylic acid recovery step.

Para. 12. The method of any one of Para. 1-11, wherein the fermentation-derived carboxylic acids comprise short-chain fatty acids with a carbon length of C₂ to C₅, medium-chain fatty acids with a carbon length of C₆ to C₉, or a mixture of any two or more thereof.

Para. 13. The method of any one of Para. 1-12, wherein the monoglycerides have greater than 90% bio-based carbon content as measured by ASTM D6866.

Para. 14. A preservative comprising a monoglyceride or a mixture thereof, wherein the monoglyceride is a short-chain monoglyceride, medium-chain monoglyceride, or a mixture of any two or more thereof, and the monoglyceride is prepared by reacting a fermentation-derived carboxylic acid with glycerol.

Para. 15. A method of preserving a foodstuff, the method comprising contacting the foodstuff with a preservative comprising a short-chain monoglyceride, a medium-chain monoglyceride, or a mixture of any two or more thereof, wherein the monoglyceride is prepared by reacting a fermentation-derived carboxylic acid with glycerol.

Para. 16. A method of using a preservative comprising using the preservative as an antimicrobial preservative, an antifungal preservative, a texture modifier, a surfactant emulsifier, a nutritional additive, a dietary supplement, or combinations thereof, wherein the preservative comprises short-chain monoglycerides, medium-chain monoglycerides, or a combination thereof, and wherein the monoglycerides are prepared by reacting carboxylic acids obtained from a fermentation process with glycerol.

Para. 17. A method of preparing monoglycerides, the method comprising: fermenting a biodegradable feedstock to produce a first fermentation broth comprising short- and medium-chain fatty acids; recovering at least a portion of the short- and medium-chain fatty acids in the fermentation broth; esterifying the fermentation-derived carboxylic acids with glycerol to produce monoglycerides; and purifying the monoglycerides to produce a product stream comprising monoglycerides, di-glycerides, triglycerides and glycerol and a by-product stream comprising unreacted glycerol, unreacted fermentation derived carboxylic acids, or a combination of any two or more thereof; recycling the by-product stream to one or more of the fermenting step and the carboxylic acid recovering step, wherein the monoglycerides are short-chain monoglycerides, medium-chain monoglycerides, or a combination of any two or more thereof.

Para. 18. The method of Para. 17, further comprising at least one of: conditioning the fermentation broth before recovering; and fractionating the fermentation-derived carboxylic acids before the esterifying.

Para. 19. The method of Para. 17 or 18, wherein the esterifying is carried out in the presence of an acid catalyst, a base catalyst, or an enzyme catalyst.

Para. 20. The method of Para. 17, 18, or 19, wherein the monoglycerides have greater than 90% bio-based carbon content as measured by ASTM D6866.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A method of preparing monoglycerides from fermentation-derived carboxylic acids, the method comprising:
esterifying the fermentation-derived carboxylic acids with glycerol to produce the monoglycerides,
wherein the monoglycerides are short-chain monoglycerides, medium-chain monoglycerides, or a combination of any two or more thereof.

2. The method of claim 1, wherein the glycerol is present in the esterifying in stoichiometric excess over the fermentation-derived carboxylic acids.

3. The method of claim 1, wherein the esterifying is carried out without a catalyst or in the presence of a catalyst.

4. The method of claim 3, wherein the esterifying is carried out in the presence of the catalyst comprising an acid catalyst.

5. The method of claim 3, wherein the esterifying is carried out in the presence of the catalyst comprising a base catalyst.

6. The method of claim 3, wherein the esterifying is carried out in the presence of the catalyst comprising an enzyme catalyst.

7. The method of claim 1 further comprising purifying the monoglycerides.

8. The method of claim 7, wherein the purifying is performed by vacuum distillation, vacuum steam distillation, molecular distillation, or a combination of any two or more thereof.

9. The method of claim 1, wherein the fermentation-derived carboxylic acids are produced in a fermentation step, the fermentation step comprising a mixed-culture fermentation or an anaerobic digestion to produce a fermentation broth.

10. The method of claim 9 further comprising recovering the fermentation-derived carboxylic acids from the fermentation broth in a carboxylic acid recovery step.

11. The method of claim 10, wherein one or more by-product streams of the purification are recycled to the fermentation step or to the carboxylic acid recovery step.

12. The method of claim 1, wherein the fermentation-derived carboxylic acids comprise short-chain fatty acids with a carbon length of C₂ to C₅, medium-chain fatty acids with a carbon length of C₆ to C₉, or a mixture of any two or more thereof.

13. The method of claim 1, wherein the monoglycerides have greater than 90% bio-based carbon content as measured by ASTM D6866.

14. A preservative comprising a monoglyceride or a mixture thereof, wherein the monoglyceride is a short-chain monoglyceride, a medium-chain monoglyceride, or a mixture of any two or more thereof, and the monoglyceride is prepared by reacting a fermentation-derived carboxylic acid with glycerol.

15. The preservative of claim 14, wherein the monoglycerides have greater than 90% bio-based carbon content as measured by ASTM D6866.

16. The preservative of claim 14, wherein the monoglyceride is prepared by reacting the fermentation-derived carboxylic acid with glycerol in the presence of an acid catalyst.

17. A method of preparing monoglycerides, the method comprising:
fermenting a biodegradable feedstock to produce a first fermentation broth comprising short- and medium-chain carboxylic acids;
recovering at least a portion of the short- and medium-chain carboxylic acids in the fermentation broth;
esterifying the fermentation-derived carboxylic acids with glycerol to produce monoglycerides; and
purifying the monoglycerides to produce a product stream comprising monoglycerides, di-glycerides, triglycerides and glycerol and a by-product stream comprising unreacted glycerol, unreacted fermentation-derived carboxylic acids, or a combination of any two or more thereof;
recycling the by-product stream to one or more of the fermenting step and the carboxylic acid recovering step,
wherein the monoglycerides are short-chain monoglycerides, medium-chain monoglycerides, or a combination of any two or more thereof.

18. The method of claim 17, further comprising at least one of:
conditioning the fermentation broth before recovering; and
fractionating the fermentation-derived carboxylic acids before the esterifying.

19. The method of claim 17, wherein the esterifying is carried out in the presence of an acid catalyst, a base catalyst, or an enzyme catalyst.

20. The method of claim 17, wherein the monoglycerides have greater than 90% bio-based carbon content as measured by ASTM D6866.
